(19) United States
(12) Plant Patent Application Publication
(10) Pub. No.: US 2013/0239270 P1
(43) Pub. Date: Sep. 12, 2013

(54) Variety of basil plant named 'Dark Lady'

(50) ;

(71) Applicant: Udo Schäfer, Wolsier (DE)

(72) Inventor: Udo Schäfer, Wolsier (DE)

(73) Assignee: Herbalea GmbH, Grolsheim (DE)

(21) Appl. No.: 13/385,798

(22) Filed: Mar. 7, 2012

(51) Int. Cl. A01H 5/00

(52) U.S. Cl. PLT/258

(57) ABSTRACT

‘Dark Lady’ is a new variety of basil plant having tolerance to low temperatures, a sweet-spicy taste, and dark violet leaves with prominent veins.

### BOTANICAL CLASSIFICATION

Ocimum basilicum.

### VARIETAL DENOMINATION

‘DARK LADY’

### BACKGROUND OF THE NEW PLANT

The present invention comprises a new and distinct cultivar of basil plant, botanically known as Ocimum basilicum and hereinafter referred to by the cultivar name ‘Dark Lady’.

The new variety was discovered and selected by the breeder in a cultivated environment in July of 2009 in Wolsier, Germany (located in the region of Brandenburg) as a naturally occurring open-pollinated new variety in a population of unpatented and unnamed Ocimum basilicum plants. Subsequently, the new variety was asexually reproduced by cuttings in Wolsier, Germany in 2010. Further asexual reproductions at the same location has demonstrated that the combination of characteristics as herein disclosed for the new cultivar are retained and reproduced true to type through successive generations of asexual reproduction.

The following observations, measurements and comparisons describe 8-week-old plants grown in an open field with full sun, watered as needed, and receiving weekly fertilization treatments accented with nitrogen in Wolsier, Germany. The plants were exposed to daytime temperatures of 20-30° C., and evening temperatures of 10-20° C. Color observations were taken under natural light, and the color references are made to the R.H.S. Colour Chart of The Royal Horticulture Society of London, except where general color terms of ordinary significance are used.

The present invention has not been evaluated under all possible environmental conditions. The phenotype may vary with variation in environment without a change in the genotype of the plant.

The following traits have been repeatedly observed and determined to basic characteristics of ‘Dark Lady’ which, in combination, distinguish this basil plant as a new and distinct cultivar:

1. Tall and well-branched plant habit;

2. Sweet-spicy fragrance;

3. Aromatic taste;

4. Tolerant to Fusarium, Botrytis, and Pythium; and

5. Low temperature tolerance.

Further, the new variety exhibits a tall and well-branched habit like basil variety ‘Ajaka’ (U.S. Plant patent applied for concurrently). However, ‘Dark Lady’ has very dark violet leaves and an aromatic fragrance, while ‘Ajaka’ has green leaves and a sweet-spicy fragrance. The following Table 1 provides a further comparative listing of botanical characteristics of ‘Dark Lady’ and ‘Ajaka’.

TABLE 1

| Botanical | | |
|---|---|---|
| characteristics | ‘Dark Lady’ | ‘Ajaka’ |
| Height from soil to | 50 cm. | 60 cm. |
| top of plant: | | |
| Leaf length: | 4.5 cm. | 5 cm. |
| Number of flowers | About 200 | About 150 |
| per plant: | | |
| Flower longevity: | 6-8 weeks | 4-6 weeks |

### BRIEF DESCRIPTION OF THE DRAWINGS

The accompanying photographic drawings illustrate the new variety, with the colors being as nearly true as is possible with color illustrations of this type:

FIG. 1 is a close-up photograph of the leaves of the new variety;

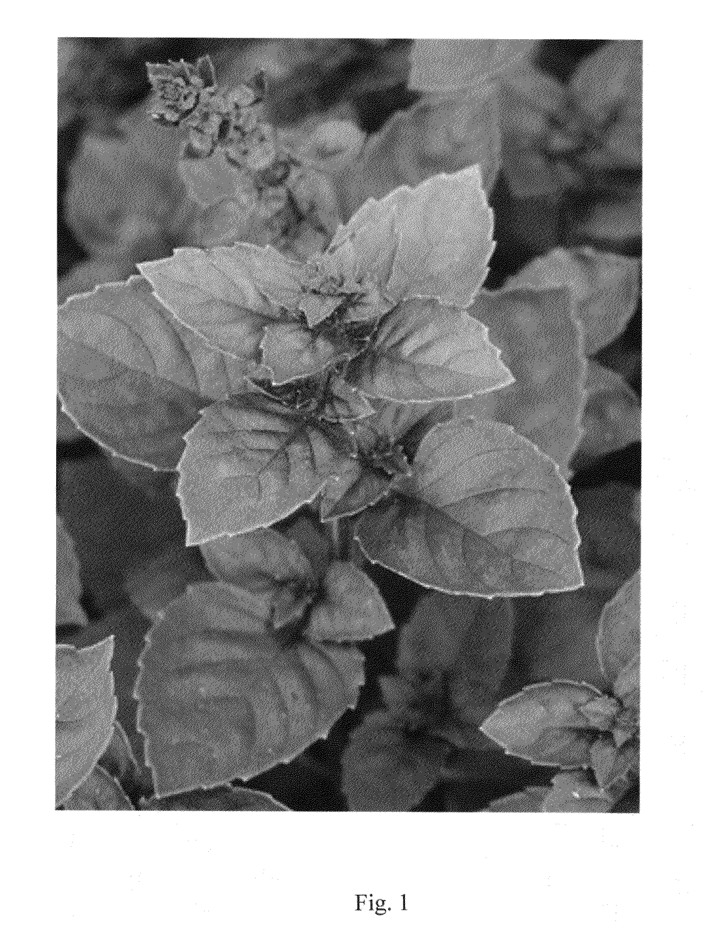

FIG. 2 is a photograph of a field of plants of the new variety;

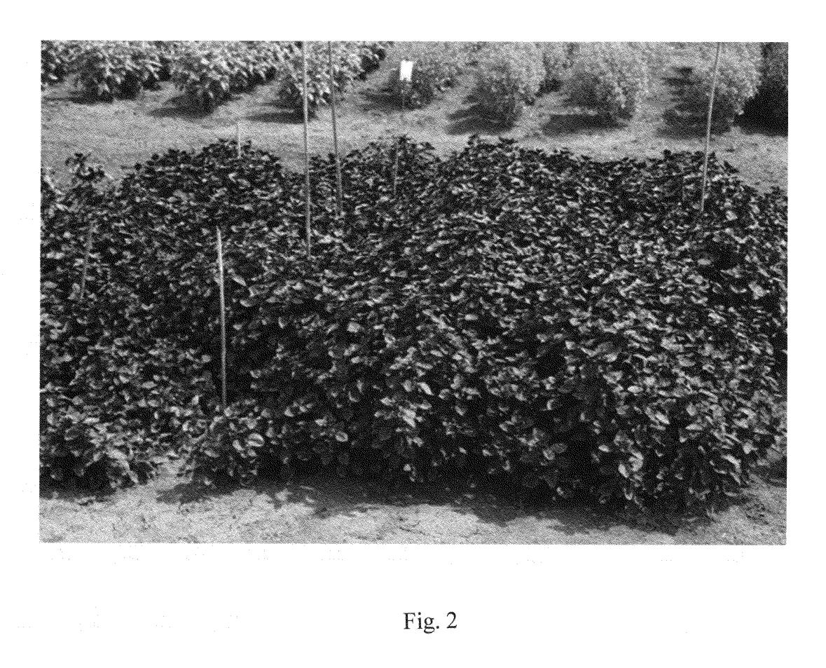

FIG. 3 is a another photograph of a field of plants of the new variety;

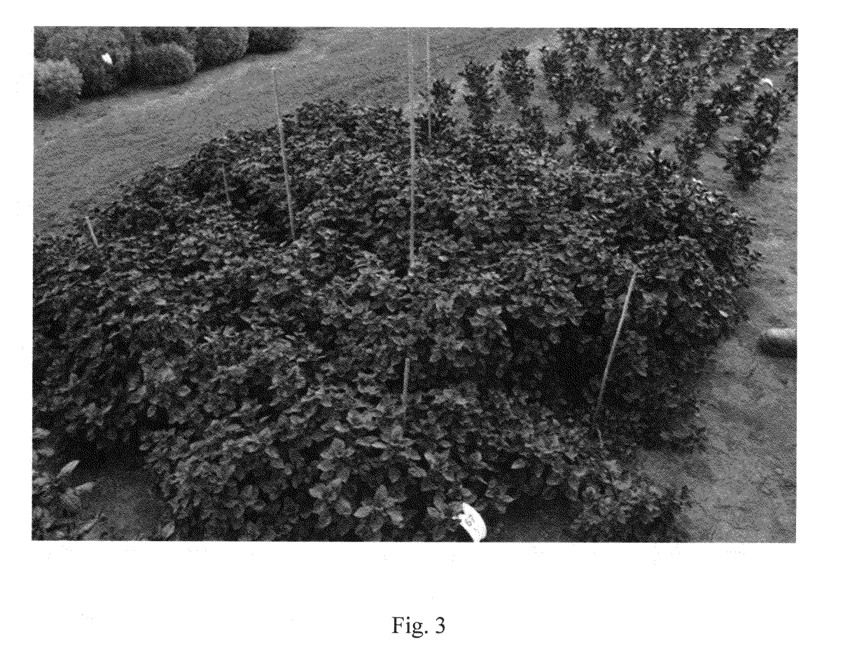

FIG. 4 is a close-up photograph of the flowers of the new variety;

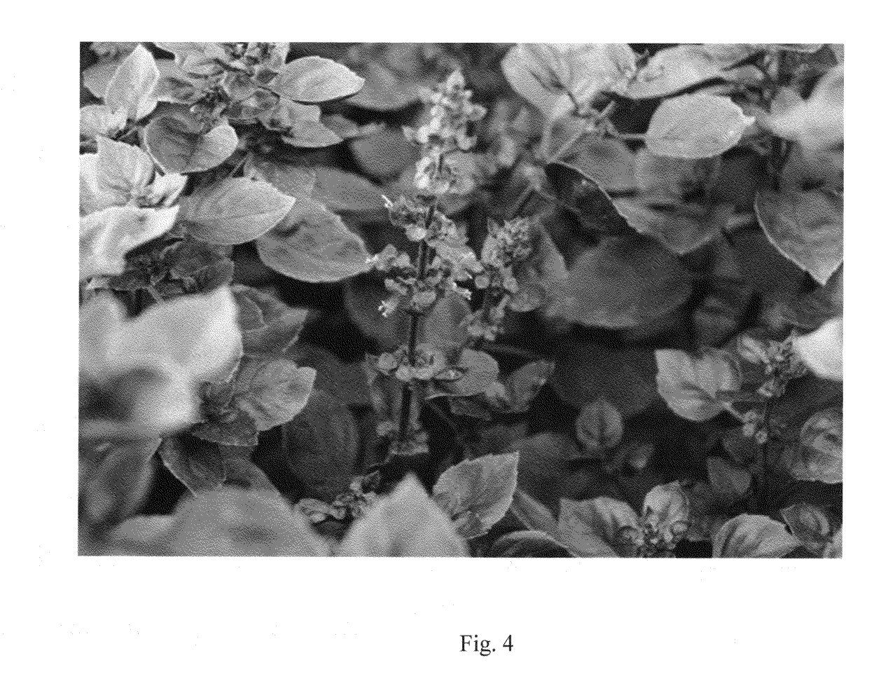

FIG. 5 is another close-up photograph of the flowers of the new variety;

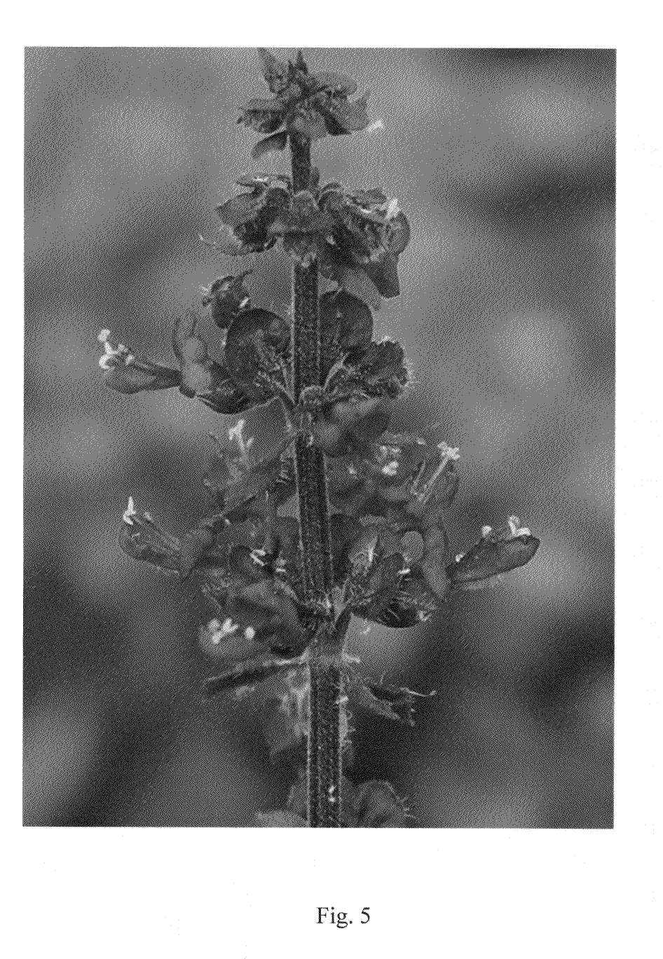

FIG. 6 is a further close-up photograph of the flowers of the new variety; and

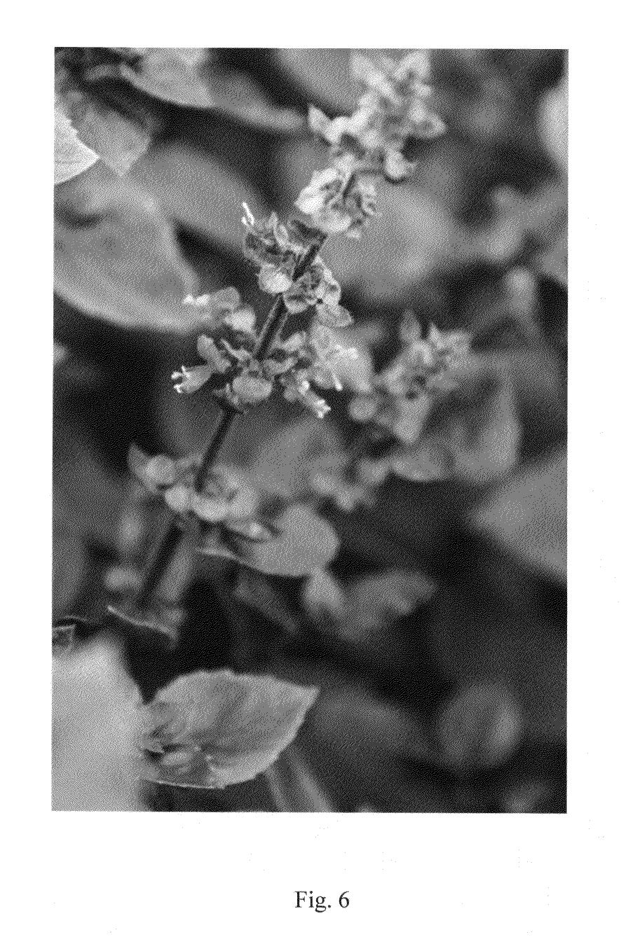

FIG. 7 is a photograph of detached leaves and flowers of the new variety.

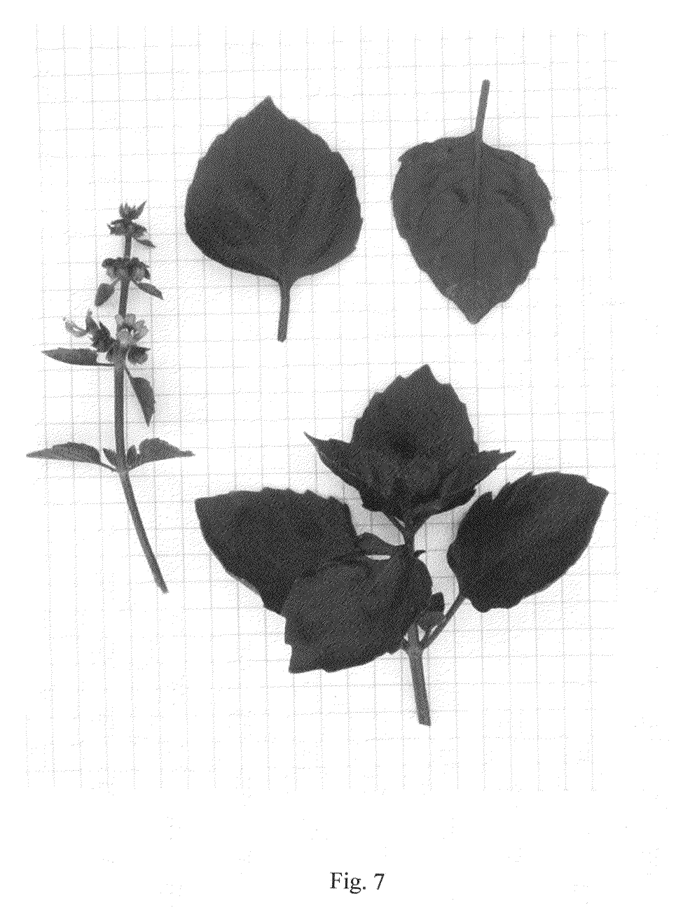

### DESCRIPTION OF THE PLANT

Time to initiate roots: 5 days at 22° C.
Time to produce a rooted plant: 9 days at 22° C.
Root description: Finely branched white roots with a fibrous habit.
Form: Erect.
Height from soil to top of plant: 50 cm.
Plant diameter: 40 cm.
Vigor: Erect and loose with a low-branching habit.
Main stem:
   Length.—50 cm.
   Width.—0.5 cm.
   Color.—Purple Group RHS N77B to Violet Group RHS 83A.
   Number of branches.—5-7.
   Node spacing.—3-5 cm.
Leaves:
   Arrangement.—Decussate; pinnate.
   Number of leaves per plant.—About 400.
   Length.—4.5 cm.
   Width.—3.5 cm.
   Shape of leaf (generally).—Broadly ovate.
   Shape of apex.—Acute.
   Shape of base.—Broadly ovate.
   Texture.—Very soft and plain on the upper surface of the leaf. The veins are very strong and visible, especially on the lower leaf surface.
   Aspect.—Horizontal.
   Margin type.—Moderate serration present.
   Upper surface color.—Violet Group RHS 83A up to a blackish-violet color.
   Lower surface color.—Violet Group RHS 83A up to a blackish-violet color.
   Veins.—Upper surface description and color: Pinnate; Violet Group RHS 83A up to a blackish-violet color.
   Lower surface description and color.—Pinnate; Violet Group RHS 83A up to a blackish-violet color.
   Petiole.—Length: 2 cm. Diameter: 2 mm. Color: Violet Group RHS 83A up to a blackish-violet color. Texture: Hairy. Shape: Round.
Reproductive organs: 1 pistil and 4 stamens present.
Flowers:
   Natural flowering season.—July through September in the Brandenburg Region of Germany.
   Number of flowers per plant.—About 200.
   Fragrance.—Aromatic.
   Longevity.—6-8 weeks.
   Flower type.—Labiate.
   Flower height.—0.5 cm.
   Flower diameter.—0.3 cm.
   Petal color.—Upper surface: Red-Purple Group RHS N74A. Lower surface: Red-Purple Group RHS N74A.
Seed description: Sterile.
Disease/pest resistance: Resistant to Botrytis cinerea and tolerant to Fusarium oxysporum.
Weather tolerance: Very tolerant to rain and cold nights (down to 8° C.).
Harvesting season: Early summer to fall outdoors; year round indoors.
Flavor: Sweet-spicy.
Aroma: Condimental.
Essential oils: Linalool-type.
Productivity/yield per acre:
   Fresh.—8 tons per acre.
   Dried.—1 tons per acre.
Average life span: 5 months.
Use: Multiple uses. The new variety can be used as a pot plant, ornamental plant, culinary herb, or fresh and dried herb.
Fresh shelf life: Over 1 week under cool conditions (5-8° C.).

I claim:

1. A new and distinct variety of basil plant named ‘Dark Lady’ as herein described and illustrated.
